(19) United States
(12) Patent Application Publication
(10) Pub. No.: US 2019/0021952 A1
(43) Pub. Date: Jan. 24, 2019

(54) Stabilized Antibody Compositions and Methods of Producing Same

(71) Applicant: Regeneron Pharmaceuticals, Inc., Tarrytown, NY (US)

(72) Inventors: Xiaolin TANG, Old Tappan, NJ (US); David Brett LUDWIG, New York, NY (US)

(21) Appl. No.: 16/042,099

(22) Filed: Jul. 23, 2018

Related U.S. Application Data
(60) Provisional application No. 62/536,028, filed on Jul. 24, 2017.

(51) Int. Cl. A61J 1/20; A61J 1/06

(52) U.S. Cl. CPC A61J 1/2068; A61K 9/0019; A61J 1/06

(57) ABSTRACT

The present invention provides drug products comprising sealed containers containing a stable liquid recombinant protein, such as an antigen-binding protein, composition and a headspace gas comprising less than 5% oxygen, and methods of producing same. As such, the present invention provides a method for controlling the oxygen levels in the headspace of a drug product in a sealed container comprising a stable liquid recombinant protein composition and a headspace gas comprising less than 21% oxygen.

### CROSS-REFERENCE TO RELATED APPLICATIONS

This application claims the benefit under 35 USC § 119(e) of U.S. Provisional Application No. 62/536,028, filed Jul. 24, 2017, which is herein incorporated by reference in its entirety.

### FIELD OF THE INVENTION

The present invention relates to liquid antibody compositions and to methods of producing such compositions by minimizing and/or controlling the level of oxidizing gases in the headspace of containers in which the compositions are filled and stored prior to administration.

### BACKGROUND

Antibody therapeutics, including monospecific and bispecific antibodies, continue to be developed for the treatment of a variety of diseases and conditions, including the treatment of cancers and autoimmune disorders. Antibody potency has increased with refinements in the production of antibody molecules directed to specific targets. Whether stored at high concentrations for small volume administration, or lower concentrations for high potency therapeutics, antibody molecules may be susceptible to oxidation when in liquid formulations filled and stored in drug product containers such as vials and administered via syringes. The importance of maintaining a stable composition to minimize losses of the biologically active agent due to oxidation or other degradative processes is emphasized by the International Conference on Harmonisation of Technical Requirements For Registration of Pharmaceuticals For Human Use (ICH). According to ICH Specifications (Q6A and Q6B), if a drug substance does not degrade in the specific formulation and under the specific storage conditions proposed in a new drug application, as demonstrated via appropriate analytical methodology, then degradation product testing may be reduced or eliminated upon approval by the regulatory authorities.

The use of nitrogen or inert gases (e.g., argon) to replace “air” in the headspace of drug product containers has been discussed in the art. EP1174148 discusses the preparation of a Fab fragment composition at a concentration of 2 mg/ml in which the gas headspace of each vial was purged with nitrogen via repeated cycles in a laboratory scale lyophilization chamber (i.e. not suitable for Good Manufacturing Practices (GMP) standards). EP1174148 neither mentions the oxygen concentration of the headspace gas following the nitrogen purge, nor provides the guidance to control the oxygen content in the headspace to a predefined target level, but significant percentage increases in the presence of high molecular weight (HMW) impurities was observed under accelerated stability storage conditions (e.g., 40° C. for 1 month). In several examples, the increase in HMW species after 1 month at 40° C. exceeded 300% (Table 1), while storage at 40° C. for three months produced an increase in HMW species of more than 14-fold (Table 4). Similarly, US 2016/0129028 discusses the use of a nitrogen overlay process to maintain stability of polysaccharides, and US 2012/0183531 discusses the reduction or replacement of oxygen in the headspace of protein drug products using nitrogen or inert gases to prevent or inhibit yellow color formation due to oxidation of histidine buffers. There is a need in the pharmaceutical industry for reliable methods that reduce the incidence or impact of degradation of drug products due to oxidation.

### BRIEF SUMMARY OF THE INVENTION

In a first aspect, the present invention provides a drug product comprising a sealed container containing a recombinant protein (e.g., an antigen-binding protein or an antibody) in a liquid formulation and a headspace comprising a gas, in which the gas comprises less than 5% oxygen by volume and the recombinant protein is stable for a period of at least 28 days when stored at 45° C. In this context, stable for at least 28 days refers to an increase in percentage of high molecular weight species of no more than 2% over the period.

In some cases, the gas comprises no more than 2% oxygen by volume, no more than 1% oxygen by volume, less than 1% oxygen by volume, or no more than 0.1% oxygen by volume.

In certain embodiments, the liquid formulation of the drug product contains the recombinant protein (e.g., antigen-binding protein) at a concentration between about 2 mg/ml to 200 mg/ml. In some embodiments, the liquid formulation of the drug product contains the recombinant protein (e.g., antigen-binding protein) at a concentration between 150-200 mg/ml. In some embodiments, the liquid formulation of the drug product contains the recombinant protein (e.g., antigen-binding protein) at a concentration of less than 10 mg/ml, less than 5 mg/ml, or less than 2 mg/ml.

In some embodiments, the drug product contains a recombinant protein that is stable for a period of at least three months when stored at 45° C., wherein stable for at least three months refers to an increase in percentage of high molecular weight species of no more than 10% over the period. In some cases, the stability of the recombinant protein refers to an increase in percentage of high molecular weight species of no more than 5% over the period, or to an increase in percentage of high molecular weight species of less than 1% over the period.

In another aspect, the present invention provides a drug product comprising a sealed container containing a recombinant protein (e.g., an antigen-binding protein or an antibody) in a liquid formulation and a headspace comprising a gas, in which the gas comprises less than 1% oxygen by volume and the recombinant protein is stable for a period of at least 31 months when stored at 5° C. In this context, stable for at least 31 months refers to a change in percentage of main charge variant species of no more than 10% over the period.

In another aspect, the present invention provides a drug product comprising a sealed container containing a recombinant protein (e.g., an antigen-binding protein) in a liquid formulation and a headspace comprising a gas, wherein the gas comprises less than 1% oxygen by volume. In some embodiments, the gas comprises no more than 0.1% oxygen by volume. In some embodiments, the recombinant protein is present in the liquid formulation at a concentration of less than 5 mg/ml. In some embodiments, the recombinant protein is present in the liquid formulation at a concentration of about 2 mg/ml. In some embodiments, the recombinant protein is present in the liquid formulation at a concentration of less than 2 mg/ml, about 1.5 mg/ml or about 1 mg/ml.

In various embodiments of the drug product discussed above or herein, the recombinant protein is an antigen-binding protein. In some cases, the antigen-binding protein is an antibody. In some cases, the antibody is a monospecific antibody. In some embodiments, the antibody is a bispecific antibody.

In some cases, the drug product container is a vial. In certain embodiments, the vial comprises a stopper, such as a rubber stopper, comprising a vent leg. In some cases, the drug product is further administered via a syringe or transferred to a syringe or injection device.

In another aspect, the present invention provides a method of preparing a drug product in a sealed container containing a recombinant protein (e.g., an antigen-binding protein or an antibody) in a liquid formulation and a headspace comprising a gas with reduced oxygen content, the method comprising: (a) loading one or more containers containing the liquid formulation of the recombinant protein into a vacuum chamber under atmospheric pressure; (b) evacuating the chamber at a first pressure of from 0.05 bar to 0.15 bar; (c) aerating the chamber with a non-oxidizing gas at a second pressure of from 800 mbar to 1000 mbar; and (d) sealing the one or more containers inside the sealed vacuum chamber, wherein the method is performed at a temperature in a range of from 15-25° C., and the sealed container comprises a headspace gas with less than 5% oxygen by volume.

In some embodiments, the method further comprises repeating steps (b) and (c) one or more additional times prior to sealing the one or more containers.

In some cases, the first pressure is about 0.1 bar, and/or the second pressure is from 0.2 bar to 0.1 bar or from 0.1 bar to 0.12 bar. In some embodiments, the pressure in the chamber is measured via a Pirani vacuum gauge. In some embodiments, the temperature is about 19° C.

In further embodiments, the stopper may be partially stoppered prior to inert gas overlay, then fully stoppered and sealed post the inert gas overlay process. In some cases, the sealed container comprises a headspace gas with less than 2% oxygen by volume, less than 1% oxygen by volume, or no more than 0.1% oxygen by volume.

In some embodiments, the liquid formulation in the drug product prepared according the methods discussed above or herein contains the recombinant protein at a concentration of 2 mg/ml to 200 mg/ml. In other embodiments, the liquid formulation in the drug product prepared according the methods discussed above or herein contains the recombinant protein at a concentration of 150-200 mg/ml. In some embodiments, the liquid formulation in the drug product prepared according the methods discussed above or herein contains the recombinant protein at a concentration of less than 10 mg/ml, less than 5 mg/ml, or less than 2 mg/ml.

In various embodiments of the methods discussed above or herein, the recombinant protein is an antigen-binding protein or an antibody. In some cases, the antibody is a monospecific antibody. In some embodiments, the antibody is a bispecific antibody.

In some cases, the drug product container used in the methods of the present invention is a vial. In further embodiments, the vial comprises a rubber stopper for lyophilized product with vent leg (that can be partially stoppered prior to inert gas overlay then fully stoppered and sealed post the inert gas overlay process.

In another aspect, the present invention provides a method of controlling oxygen content in a headspace of a sealed container containing a liquid pharmaceutical formulation, wherein the method comprises: (a) determining a desired final oxygen content in the headspace of the sealed container; (b) calculating an end % oxygen content following a first cycle of oxygen reduction via equation (I):

$$\% {O}_{2 , end} = ( \frac{{P}_{vacuum}}{{P}_{aeration}} ) * \% {O}_{2 , start} \qquad ( I )$$

wherein % O_{2, start} is the % oxygen content at the start of the first cycle, P_{vacuum} is an evacuation pressure applied in the first cycle of oxygen reduction, Pₐₑᵣₐₜᵢₒₙ is a pressure higher than P_{vaccum} but less than 1 bar, and % O_{2. end} is the % oxygen content at the end of the first cycle; (c) optionally applying equation (I) to additional cycles, wherein % O_{2, start} is the % oxygen content at the end of the preceding cycle, until the desired final oxygen content is reached; and (d) preparing a drug product in the sealed container by: (i) performing one or more cycles of oxygen reduction by evacuating an unsealed container in a vacuum chamber at P_{vacuum}, wherein P_{vacuum} is a pressure of from 0.05 bar to 0.15 bar, and aerating the unsealed container in the vacuum chamber with a non-oxidizing gas at an aeration pressure of from 800 mbar to 1000 mbar; and (ii) sealing the container inside the sealed lyophlization chamber.
If the requirement for the % oxygen in the headspace of drug product is low (e.g., below approximately 2%), multiple oxygen reduction cycles are performed in order to achieve the target level of oxygen content. The % oxygen content after multiple oxygen reduction cycles using the same vacuum pressure and aeration pressure is defined via equation (II).

$$\% {O}_{2 , final} = {( \frac{{P}_{vacuum}}{{P}_{areation}} )}^{n} * \% {O}_{2 , start} \qquad ( II )$$

wherein % O_{2, start} is the % oxygen content at the start of the first cycle, P_{vacuum} is an evacuation pressure applied in the cycle of oxygen reduction, Pₐₑᵣₐₜᵢₒₙ is the pressure of aeration with the inert gas, and % O_{2. final} is the % oxygen content at the end of the multiple cycles; and n is the number of total oxygen reduction cycles applied to the product. Therefore, the number of oxygen reduction cycles required to achieve the final oxygen level in the headspace of the vial is acquired by solve equation (II).

In various embodiments of the methods discussed above or herein, the non-oxidizing gas is selected from the group consisting of nitrogen, argon, helium, xenon, neon, krypton and radon. In one embodiment, the non-oxidizing gas is nitrogen. In one embodiment, the non-oxidizing gas is argon.

The various embodiments discussed above or herein may be combined in any manner consistent with the present invention. Other embodiments will become apparent from a review of the ensuing detailed description.

### BRIEF DESCRIPTION OF THE DRAWINGS

FIG. 1 illustrates the % change in main charge variant species by CEX-UPLC as a function of oxygen headspace content for a liquid formulation of a bispecific antibody following 31 months of storage at 5° C.

FIG. 2 illustrates the % increase in high molecular weight species as function of oxygen headspace content for a liquid formulation of a bispecific antibody following 28 days of storage at 45° C. using a nitrogen overlay in accordance with the methods discussed herein.

FIG. 3 illustrates the % increase in high molecular weight species as function of oxygen headspace content for a liquid formulation of a bispecific antibody following 3 months of storage at 45° C. using a nitrogen overlay in accordance with the methods discussed herein.

FIG. 4 illustrates the % increase in high molecular weight species as function of oxygen headspace content for a liquid formulation of a bispecific antibody following 3 months of storage at 45° C. using an argon overlay in accordance with the methods discussed herein.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present invention, the preferred methods and materials are now described. All patents, applications and non-patent publications mentioned in this specification are incorporated herein by reference in their entireties.

### Definitions

The term “recombinant protein”, as used herein, is intended to include all proteins that are prepared, expressed, created or isolated by recombinant means, such as proteins expressed using a recombinant expression vector transfected into a host cell.

The term “antigen-binding molecule” or “antigen-binding protein” includes antibodies and antigen-binding fragments of antibodies, including, e.g., monospecific and bispecific antibodies.

The term “antibody”, as used herein, means any antigen-binding molecule or molecular complex comprising at least one complementarity determining region (CDR) that specifically binds to or interacts with a particular antigen. The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the antibody (or antigen-binding portion thereof) may be identical to the human germline sequences, or may be naturally or artificially modified. An amino acid consensus sequence may be defined based on a side-by-side analysis of two or more CDRs.

The term “antibody”, as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}—V_{H}, V_{H}—V_{L} or V_{L}—V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (V) V_{H}-C_{H}1-C_{H}2-C_{H}3; V_{H}-C_{H}2-C_{H}3; (Vii) V_{H}-C_{L}; V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the present invention using routine techniques available in the art.

The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The antibodies of the invention may, in some embodiments, be recombinant human antibodies. The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The instant invention encompasses antibodies having one or more mutations in the hinge, C_{H}2 or C_{H}3 region which may be desirable, for example, in production, to improve the yield of the desired antibody form.

The antibodies of the invention may be isolated antibodies. An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present invention. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The present invention also includes one-arm antibodies that bind a specific antigen. As used herein, a “one-arm antibody” means an antigen-binding molecule comprising a single antibody heavy chain and a single antibody light chain.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, and more preferably at least about 96%, 97%, 98% or 99% of the nucleotide bases, as measured by any well-known algorithm of sequence identity, such as FASTA, BLAST or Gap, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402, each herein incorporated by reference.

### Drug Products and Compositions Comprising a Recombinant Protein

Drug products of the present invention comprise a sealed container (e.g., a vial) in which the gas in the headspace has a reduced concentration of an oxidizing gas (e.g., oxygen) compared to atmospheric concentrations of the oxidizing gas. The drug products of the present invention are based on the inventors' discovery of a method for reducing the level of oxidizing gases in the headspace of a drug product container that contains a liquid pharmaceutical formulation of a recombinant protein (e.g., an antigen-binding protein or an antibody). In contrast to standard lyophilization techniques, evacuation and aeration of the headspace gas over a liquid composition requires fine control of the pressure in the vacuum chamber in order to minimize or eliminate bubbling or splashing of the liquid composition, which may cause loss of material, or evaporation, which may result in a change in the concentration of the active agent (e.g., antibody). The loss of material or change in concentration is particularly problematic for high potency compositions in which the active agent is present at low concentrations (e.g., about 2 mg/ml). Changes in stability of high concentration formulations are problematic since oxidation degradation products may result in a complex purity/impurity profile, possibly leading to immunogenicity concerns.

The drug products of the present invention may contain a headspace gas with less than 5% of an oxidizing gas (e.g., oxygen) by volume in some embodiments. The concentration of oxidizing gas (e.g., oxygen) in the headspace of the drug product container may be less than 4.5%, less than 4%, less than 3.5%, less than 3%, less than 2.5%, less than 2%, or less than 1.5% in various embodiments. In one embodiment, the concentration of the oxidizing gas (e.g., oxygen) in the headspace is less than about 1%. In one embodiment, the concentration of the oxidizing gas (e.g. oxygen) in the headspace is no more than about 0.5%. In one embodiment, the concentration of the oxidizing gas (e.g. oxygen) in the headspace is no more than about 0.1%. In various embodiments, the concentration of the oxidizing gas (e.g., oxygen) in the headspace of the drug product container is less than 0.9%, less than 0.8%, less than 0.7%, less than 0.6%, less than 0.5%, less than 0.4%, less than 0.3%, less than 0.2%, or less than 0.1%. In some cases, the concentration of oxygen in the headspace gas is from about 0.01% to about 1.5%. In some cases, the concentration of oxygen in the headspace gas is from about 0.75% to about 1.25%. In some cases, the concentration of oxygen in the headspace gas is from about 0.05% to about 0.15%.

The container described herein can be a vial, flask, etc., with sufficient volume to accommodate the desired amount of pharmaceutical formulation and headspace. The container can be formed from a variety of suitable materials which exhibit inert characteristics with respect to the pharmaceutical formulation to be contained therein, and sufficiently impermeable to prevent leakage of the pharmaceutical formulation, or infiltration of ambient air. Exemplary materials include glass (e.g. Polycarbonate Polystyrene Polypropylene Glass), polymers (e.g., plastic, Platinum Cured Silicone Tubing) and metals (e.g., stainless steel 316L). In some embodiments, the container is a type 1 glass vial. Additionally, the container can be configured as a reusable component, or a single-use disposable component, as so desired.

Multiple designs for rubber stops with vent leg(s) are available and suitable for use with a lyophilzation vial adapted for use in the methods described herein. A stopper comprising one vent leg (single vent), ‘two-leg’ (two vent points), ‘three-leg’ (three vent points), and cruciform (four vent-points) or even more vents, are commercially available. A stopper for use in a vial in a lyophilization chamber may be partially stoppered with the vent(s) open to the outside airspace during the gas overlay/oxygen reduction process, and then fully stoppered and sealed in connection with the container following the one or more oxygen reduction cycles.

The drug products of present invention include liquid pharmaceutical compositions comprising the antigen-binding molecules (e.g., an antibody) of the present invention. The pharmaceutical compositions of the invention are formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, Pa. For example, the excipients can include a stabilizer, a buffer, a tonicifier, a surfactant, an organic solvent, a salt or a combination thereof. In some embodiments the stabilizer is selected from the group consisting of a polyol, a sugar, an amino acid, a non-ionic surfactant, and a combination thereof. In some embodiments the tonicifier is selected from the group consisting of a sugar, an amino acid, a salt, and a combination thereof. In some embodiments the buffer is selected from the group consisting of histidine, phosphate, citrate, succinate, acetate, carbonate, and a combination thereof.

The concentration of the antigen-binding molecules (e.g., antibody) in the liquid compositions may vary depending on the potency of the molecules and the dose to be administered from the drug product container. In some cases, the concentration can range from about 1 mg/ml to about 200 mg/ml. In some cases, the concentration can range from about 1 mg/ml to about 10 mg/ml. In some cases, the concentration can range from about 1 mg/ml to about 5 mg/ml. In some cases, the concentration can range from about 0.1 mg/ml to about 2 mg/ml. In various embodiments, the concentration is less than about 25 mg/ml, less than about 20 mg/ml, less than about 15 mg/ml, less than about 10 mg/ml, or less than about 5 mg/ml. In various embodiments, the concentration of the antigen-binding molecules in the liquid composition is no more than about 5 mg/ml, no more than about 4 mg/ml, no more than about 3 mg/ml, no more than about 2 mg/ml, or no more than about 1 mg/ml. In one embodiment, the concentration is less than about 2 mg/ml. In one embodiment, the concentration is less than about 1 mg/ml.

The concentration of the antigen-binding molecules (e.g., antibody) in the liquid compositions may vary depending on the volume and the dose to be administered from the drug product container. In some cases, the concentration can range from about 1 mg/ml to about 200 mg/ml. In some cases, the concentration can range from about 10 mg/ml to about 200 mg/ml. In some cases, the concentration can range from about 50 mg/ml to about 100 mg/ml. In some cases, the concentration can range from about 100 mg/ml to about 150 mg/ml. In some cases, the concentration can range from about 150 mg/ml to about 200 mg/ml. In one embodiment, the concentration is greater than about 10 mg/ml. In another embodiment, the concentration is greater than about 50 mg/ml. In another embodiment, the concentration is less than about 100 mg/ml. In one embodiment, the concentration is greater than about 150 mg/ml.

The dose of antigen-binding molecule administered to a patient may vary depending upon the age and the size of the patient, target disease, conditions, route of administration, and the like. The preferred dose is typically calculated according to body weight or body surface area. When an antigen-binding molecule of the present invention is used for therapeutic purposes in an adult patient, it may be advantageous to intravenously administer the antigen-binding molecule of the present invention normally at a single dose of about 0.01 to about 20 mg/kg body weight, more preferably about 0.02 to about 7, about 0.03 to about 5, or about 0.05 to about 3 mg/kg body weight. Depending on the severity of the condition, the frequency and the duration of the treatment can be adjusted. Effective dosages and schedules for administering an antigen-binding molecule may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

The injectable liquid preparations may include dosage forms for intravenous, subcutaneous, intracutaneous and intramuscular injections, drip infusions, etc. These injectable preparations may be prepared by methods publicly known. For example, the injectable preparations may be prepared in a sterile aqueous medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. The injection thus prepared may be filled in or transferred to an appropriate container or device (e.g., an ampoule, a syringe, an injection device or pen).

### Stability of the Antigen-Binding Molecules

Reduction of the amount of oxidizing gas (e.g., oxygen) in the headspace of the drug product containers of the present invention advantageously impacts the stability of the antigen-binding molecules formulated in the liquid compositions within the containers. Oxidation is a major degradation pathway of protein therapeutics, including antibodies and bispecific antigen-binding molecules. The impact of such degradation is pronounced at low concentrations, when any loss of active material has a disproportionately greater effect on the amount of active agent remaining in the compositions after a defined period of storage. Manifestations of such degradation include increases in high molecular weight (HMW) species and changes in the percentages of charge variant species. Changes in the quantity or percentage of HMW species can be detected using standard size exclusion chromatography techniques known in the art (e.g., Lu et al., MAbs, 5(1):102-113, 2013). Changes in the quantity or percentage of charge variant species can be detected using standard cation exchange chromatography techniques known in the art (e.g., Chumsae, et al., Journal of Chromatography B, 850:285-294, 2007). Stability of the antigen-binding molecules in the drug products of the present invention can be ascertained by measuring changes in the quantity or percentage of HMW or charge variant species as a function of time and temperature parameters corresponding to specific storage conditions. In some cases, the storage conditions may be comparable to those conditions under which the drug product would ordinarily be maintained between manufacturing and use. In other cases, the storage conditions may be accelerated conditions meant to obtain an indication of longer-term stability in a shorter period of time.

In various embodiments of the compositions of the present invention, the antigen-binding molecules (e.g., antibodies) remain stable for a period of at least 28 days when stored at 45° C. Stability, in this context, can refer, for example, to an increase in percentage of HMW species of no more than about 2% over the period of storage. In some cases, the percentage increase in HMW species is no more than about 1.5%, or no more than about 1% over the period of storage. In some cases, the antigen-binding molecules remain stable for a period of at least three months when stored at 45° C. Under these longer storage conditions, stability can refer, e.g., to an increase in HMW species of no more than about 10% over the period of storage. In some cases, stability under these longer storage conditions can refer to an increase in HMW species of no more than about 5%, no more than about 4%, no more than about 3%, no more than about 2%, or no more than about 1% over the storage period. In other embodiments, the antigen-binding molecules (e.g., antibodies) remain stable for a period of at least 31 months when stored at 5° C. Stability, in this context, can refer, for example, to a change in percentage of charge variant species of no more than 10% over the period of storage. In some cases, the period of storage can be 12 months, 18 months, 24 months, 30 months or 36 months.

Throughout the process of manufacturing a particular therapeutic protein product, certain product quality attributes may be identified based upon their potential clinical impact. Relevant quality attributes may be deemed critical quality attributes (CQAs) depending upon their potential impact on purity, safety and/or efficacy. High molecular weight (HMW) species and charge variants are just two of many product CQAs that may be altered during the manufacturing process. Proteins are monitored for changes in these quality attributes during the manufacturing process, including after filling the product into a container and during container storage. A drug product that is sensitive to oxidation refers to changes in a CQA of the product above or below a threshold for that particular CQA due to increased oxidation levels which may affect the purity, safety and/or efficacy of the product. A drug product that is sensitive to oxidation also refers to changes in the product that may affect the purity, safety and/or efficacy of the product due to increased oxidation levels. In some cases, stability can refer to changes in the product CQAs above or below a predetermined threshold that may affect the purity, safety and/or efficacy of the product.

### Binding Properties of the Antigen-Binding Molecules

As used herein, the term “binding” in the context of the binding of an antigen-binding molecule, antibody, immunoglobulin, antibody-binding fragment, or Fc-containing protein to either, e.g., a predetermined antigen, such as a cell surface protein or fragment thereof, typically refers to an interaction or association between a minimum of two entities or molecular structures, such as an antibody-antigen interaction.

For instance, binding affinity typically corresponds to a K_{D} value of about 10⁻⁷ M or less, such as about 10⁻⁸ M or less, such as about 10⁻⁹ M or less when determined by, for instance, surface plasmon resonance (SPR) technology in a BIAcore 3000 instrument using the antigen as the ligand and the antibody, Ig, antibody-binding fragment, or Fc-containing protein as the analyte (or antiligand). Cell-based binding strategies, such as fluorescent-activated cell sorting (FACS) binding assays, are also routinely used, and FACS data correlates well with other methods such as radioligand competition binding and SPR (Benedict, C A, J Immunol Methods. 1997, 201(2):223-31; Geuijen, C A, et al. J Immunol Methods. 2005, 302(1-2):68-77).

Accordingly, the antibody or antigen-binding protein of the invention binds to the predetermined antigen or cell surface molecule (receptor) having an affinity corresponding to a K_{D} value that is at least ten-fold lower than its affinity for binding to a non-specific antigen (e.g., BSA, casein). According to the present invention, the affinity of an antibody corresponding to a K_{D} value that is equal to or less than ten-fold lower than a non-specific antigen may be considered non-detectable binding, however such an antibody may be paired with a second antigen binding arm for the production of a bispecific antibody of the invention.

The term “K_{D}” (M) refers to the dissociation equilibrium constant of a particular antibody-antigen interaction, or the dissociation equilibrium constant of an antibody or antibody-binding fragment binding to an antigen. There is an inverse relationship between K_{D} and binding affinity, therefore the smaller the K_{D} value, the higher, i.e. stronger, the affinity. Thus, the terms “higher affinity” or “stronger affinity” relate to a higher ability to form an interaction and therefore a smaller K_{D} value, and conversely the terms “lower affinity” or “weaker affinity” relate to a lower ability to form an interaction and therefore a larger K_{D} value. In some circumstances, a higher binding affinity (or K_{D}) of a particular molecule (e.g. antibody) to its interactive partner molecule (e.g. antigen X) compared to the binding affinity of the molecule (e.g. antibody) to another interactive partner molecule (e.g. antigen Y) may be expressed as a binding ratio determined by dividing the larger K_{D} value (lower, or weaker, affinity) by the smaller K_{D} (higher, or stronger, affinity), for example expressed as 5-fold or 10-fold greater binding affinity, as the case may be.

The term “k_{d}” (sec−1 or 1/s) refers to the dissociation rate constant of a particular antibody-antigen interaction, or the dissociation rate constant of an antibody or antibody-binding fragment. Said value is also referred to as the k_{off} value.

The term “kₐ” (M−1×sec−1 or 1/M) refers to the association rate constant of a particular antibody-antigen interaction, or the association rate constant of an antibody or antibody-binding fragment.

The term “K_{A}” (M−1 or 1/M) refers to the association equilibrium constant of a particular antibody-antigen interaction, or the association equilibrium constant of an antibody or antibody-binding fragment. The association equilibrium constant is obtained by dividing the kₐ by the k_{d}.

The term “EC50” or “EC₅₀” refers to the half maximal effective concentration, which includes the concentration of an antibody which induces a response halfway between the baseline and maximum after a specified exposure time. The EC₅₀ essentially represents the concentration of an antibody where 50% of its maximal effect is observed. In certain embodiments, the EC₅₀ value equals the concentration of an antibody of the invention that gives half-maximal binding to cells expressing CD3 or tumor-associated antigen, as determined by e.g. a FACS binding assay. Thus, reduced or weaker binding is observed with an increased EC₅₀, or half maximal effective concentration value.

In one embodiment, decreased binding can be defined as an increased EC₅₀ antibody concentration which enables binding to the half-maximal amount of target cells.

In another embodiment, the EC₅₀ value represents the concentration of an antibody of the invention that elicits half-maximal depletion of target cells by T cell cytotoxic activity. Thus, increased cytotoxic activity (e.g. T cell-mediated tumor cell killing) is observed with a decreased EC₅₀, or half maximal effective concentration value.

### Bispecific Antigen-Binding Molecules

The antigen-binding molecules, e.g., antibodies, of the present invention may be monospecific, bi-specific, or multispecific. Multispecific antibodies may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244. The antibodies of the present invention can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multispecific antibody with a second or additional binding specificity.

As used herein, the expression “antigen-binding molecule” means a protein, polypeptide or molecular complex comprising or consisting of at least one complementarity determining region (CDR) that alone, or in combination with one or more additional CDRs and/or framework regions (FRs), specifically binds to a particular antigen. In certain embodiments, an antigen-binding molecule is an antibody or a fragment of an antibody, as those terms are defined elsewhere herein.

As used herein, the expression “bispecific antigen-binding molecule” means a protein, polypeptide or molecular complex comprising at least a first antigen-binding domain and a second antigen-binding domain. Each antigen-binding domain within the bispecific antigen-binding molecule comprises at least one CDR that alone, or in combination with one or more additional CDRs and/or FRs, specifically binds to a particular antigen.

In certain exemplary embodiments of the present invention, the bispecific antigen-binding molecule is a bispecific antibody. Each antigen-binding domain of a bispecific antibody comprises a heavy chain variable domain (HCVR) and a light chain variable domain (LCVR). In the context of a bispecific antigen-binding molecule comprising a first and a second antigen-binding domain (e.g., a bispecific antibody), the CDRs of the first antigen-binding domain may be designated with the prefix “A1” and the CDRs of the second antigen-binding domain may be designated with the prefix “A2”. Thus, the CDRs of the first antigen-binding domain may be referred to herein as A1-HCDR1, A1-HCDR2, and A1-HCDR3; and the CDRs of the second antigen-binding domain may be referred to herein as A2-HCDR1, A2-HCDR2, and A2-HCDR3.

The first antigen-binding domain and the second antigen-binding domain may be directly or indirectly connected to one another to form a bispecific antigen-binding molecule of the present invention. Alternatively, the first antigen-binding domain and the second antigen-binding domain may each be connected to a separate multimerizing domain. The association of one multimerizing domain with another multimerizing domain facilitates the association between the two antigen-binding domains, thereby forming a bispecific antigen-binding molecule. As used herein, a “multimerizing domain” is any macromolecule, protein, polypeptide, peptide, or amino acid that has the ability to associate with a second multimerizing domain of the same or similar structure or constitution. For example, a multimerizing domain may be a polypeptide comprising an immunoglobulin C_{H}3 domain. A non-limiting example of a multimerizing component is an Fc portion of an immunoglobulin (comprising a C_{H}2-C_{H}3 domain), e.g., an Fc domain of an IgG selected from the isotypes IgG1, IgG2, IgG3, and IgG4, as well as any allotype within each isotype group.

Bispecific antigen-binding molecules of the present invention will typically comprise two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

In certain embodiments, the multimerizing domain is an Fc fragment or an amino acid sequence of from 1 to about 200 amino acids in length containing at least one cysteine residue. In other embodiments, the multimerizing domain is a cysteine residue, or a short cysteine-containing peptide. Other multimerizing domains include peptides or polypeptides comprising or consisting of a leucine zipper, a helix-loop motif, or a coiled-coil motif.

Any bispecific antibody format or technology may be used to make the bispecific antigen-binding molecules of the present invention. For example, an antibody or fragment thereof having a first antigen binding specificity can be functionally linked (e.g., by chemical coupling, genetic fusion, noncovalent association or otherwise) to one or more other molecular entities, such as another antibody or antibody fragment having a second antigen-binding specificity to produce a bispecific antigen-binding molecule. Specific exemplary bispecific formats that can be used in the context of the present invention include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mabe bispecific formats (see, e.g., Klein et al. 2012, mAbs 4:6, 1-11, and references cited therein, for a review of the foregoing formats).

In the context of bispecific antigen-binding molecules of the present invention, the multimerizing domains, e.g., Fc domains, may comprise one or more amino acid changes (e.g., insertions, deletions or substitutions) as compared to the wild-type, naturally occurring version of the Fc domain. For example, the invention includes bispecific antigen-binding molecules comprising one or more modifications in the Fc domain that results in a modified Fc domain having a modified binding interaction (e.g., enhanced or diminished) between Fc and FcRn. In one embodiment, the bispecific antigen-binding molecule comprises a modification in a C_{H}2 or a C_{H}3 region, wherein the modification increases the affinity of the Fc domain to FcRn in an acidic environment (e.g., in an endosome where pH ranges from about 5.5 to about 6.0). Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 259I (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P).

The present invention also includes bispecific antigen-binding molecules comprising a first C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). See, for example, U.S. Pat. No. 8,586,713. Further modifications that may be found within the second C_{H}3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies.

In certain embodiments, the Fc domain may be chimeric, combining Fc sequences derived from more than one immunoglobulin isotype. For example, a chimeric Fc domain can comprise part or all of a C_{H}2 sequence derived from a human IgG1, human IgG2 or human IgG4 C_{H}2 region, and part or all of a C_{H}3 sequence derived from a human IgG1, human IgG2 or human IgG4. A chimeric Fc domain can also contain a chimeric hinge region. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric Fc domain that can be included in any of the antigen-binding molecules set forth herein comprises, from N- to C-terminus: [IgG4 C_{H}1]-[IgG4 upper hinge]-[IgG2 lower hinge]-[IgG4 CH2]-[IgG4 CH3]. Another example of a chimeric Fc domain that can be included in any of the antigen-binding molecules set forth herein comprises, from N- to C-terminus: [IgG1 C_{H}1]-[IgG1 upper hinge]-[IgG2 lower hinge]-[IgG4 CH2]-[IgG1 CH3]. These and other examples of chimeric Fc domains that can be included in any of the antigen-binding molecules of the present invention are described in US Publication 2014/0243504, published Aug. 28, 2014, which is herein incorporated in its entirety. Chimeric Fc domains having these general structural arrangements, and variants thereof, can have altered Fc receptor binding, which in turn affects Fc effector function.

pH-Dependent Binding

The present invention includes antibodies and bispecific antigen-binding molecules with pH-dependent binding characteristics. For example, an antibody of the present invention may exhibit reduced binding to an antigen at acidic pH as compared to neutral pH. Alternatively, antibodies of the invention may exhibit enhanced binding to the antigen at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding . . . at acidic pH as compared to neutral pH” is expressed in terms of a ratio of the K_{D} value of the antibody binding to its antigen at acidic pH to the K_{D} value of the antibody binding to its antigen at neutral pH (or vice versa). For example, an antibody or antigen-binding fragment thereof may be regarded as exhibiting “reduced binding to MUC16 at acidic pH as compared to neutral pH” for purposes of the present invention if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment of the present invention can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0. 25.0, 30.0, 40.0, 50.0, 60.0, 70.0, 100.0 or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained.

### Antibodies Comprising Fc Variants

According to certain embodiments of the present invention, the antibodies and bispecific antigen-binding molecules include an Fc domain comprising one or more mutations which enhance or diminish antibody binding to the FcRn receptor, e.g., at acidic pH as compared to neutral pH. For example, the present invention includes antibodies comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 259I (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P).

For example, the present invention includes antibodies and bispecific antigen-binding molecules including an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); and 433K and 434F (e.g., H433K and N434F). All possible combinations of the foregoing Fc domain mutations, and other mutations within the antibody variable domains disclosed herein, are contemplated within the scope of the present invention.

### Preparation of Antigen-Binding Domains and Construction of Bispecific Molecules

Antigen-binding domains specific for particular antigens can be prepared by any antibody generating technology known in the art. Once obtained, two different antigen-binding domains, specific for two different antigens can be appropriately arranged relative to one another to produce a bispecific antigen-binding molecule of the present invention using routine methods. In certain embodiments, one or more of the individual components (e.g., heavy and light chains) of the multispecific antigen-binding molecules of the invention are derived from chimeric, humanized or fully human antibodies. Methods for making such antibodies are well known in the art. For example, one or more of the heavy and/or light chains of the bispecific antigen-binding molecules of the present invention can be prepared using VELOCIMMUNE™ technology. Using VELOCIMMUNE™ technology (or any other human antibody generating technology), high affinity chimeric antibodies to a particular antigen are initially isolated having a human variable region and a mouse constant region. The antibodies are characterized and selected for desirable characteristics, including affinity, selectivity, epitope, etc. The mouse constant regions are replaced with a desired human constant region to generate fully human heavy and/or light chains that can be incorporated into the bispecific antigen-binding molecules of the present invention.

Genetically engineered animals may be used to make human bispecific antigen-binding molecules. For example, a genetically modified mouse can be used which is incapable of rearranging and expressing an endogenous mouse immunoglobulin light chain variable sequence, wherein the mouse expresses only one or two human light chain variable domains encoded by human immunoglobulin sequences operably linked to the mouse kappa constant gene at the endogenous mouse kappa locus. Such genetically modified mice can be used to produce fully human bispecific antigen-binding molecules comprising two different heavy chains that associate with an identical light chain that comprises a variable domain derived from one of two different human light chain variable region gene segments. (See, e.g., US 2011/0195454). Fully human refers to an antibody, or antigen-binding fragment or immunoglobulin domain thereof, comprising an amino acid sequence encoded by a DNA derived from a human sequence over the entire length of each polypeptide of the antibody or antigen-binding fragment or immunoglobulin domain thereof. In some instances, the fully human sequence is derived from a protein endogenous to a human. In other instances, the fully human protein or protein sequence comprises a chimeric sequence wherein each component sequence is derived from human sequence. While not being bound by any one theory, chimeric proteins or chimeric sequences are generally designed to minimize the creation of immunogenic epitopes in the junctions of component sequences, e.g. compared to any wild-type human immunoglobulin regions or domains.

### Methods of Reducing Oxidizing Gases in the Headspace of Drug Product Containers

Methods of the present invention provide drug products with greater stability and shelf-life by minimizing charge variants and/or aggregates caused by oxidative degradation. Methods of the present invention include evacuation of the gas in the headspace of drug product containers and the subsequent aeration of the headspace with a non-oxidizing gas (e.g., nitrogen or argon) to reduce the concentration of oxygen and/or other oxidizing gases, such as ozone, peroxides, chlorine, fluorine, nitric oxide, nitrogen dioxide, nitrous oxide, or combinations thereof. The methods can be performed in a vacuum chamber (e.g., a lyophilization chamber). In one embodiment, the vacuum chamber is fitted with a Pirani vacuum gauge (thermal conductivity gauge) to accurately measure and thereby control the pressure within the ranges identified herein. In various embodiments, the methods are performed under aseptic conditions and/or under conditions that satisfy good manufacturing practice (GMP) standards for pharmaceutical drug product production.

The methods of the present invention can be used to prepare drug products in a sealed container containing a recombinant protein or an antigen-binding protein (e.g., an antibody or bispecific antigen-binding molecule) in a liquid formulation. The drug products are prepared to contain a reduced concentration of oxygen and/or other oxidizing gases in the headspace of the drug product container. Methods of preparing a drug product in a sealed container in accordance with the present invention include (a) loading one or more containers containing a liquid formulation of a recombinant protein or an antigen-binding protein (e.g., an antibody) into a vacuum chamber under atmospheric pressure, (b) evacuating the chamber at a first pressure of from about 0.05 bar to about 0.15 bar, (c) aerating the chamber with a non-oxidizing gas at a second pressure of from about 800 mbar to about 1000 mbar, and (d) sealing the one or more containers inside the sealed vacuum chamber. In some embodiments, the process steps (b) and (c) are repeated one or more times prior to sealing the container(s) to further reduce the concentration of oxidizing gases in the headspace. In various embodiments, methods of the present invention can be used to produce drug products with less than 5% oxygen (or other oxidizing gases) by volume in the container headspace. In some cases, the oxidizing gas concentration is reduced to less than 4%, less than 3%, less than 2%, or less than 1%. In some embodiments, the oxidizing gas (e.g., oxygen) concentration is no more than 0.5%, no more than 0.4%, no more than 0.3%, no more than 0.2%, or no more than 0.1%.

In some embodiments, the final desired concentration of oxygen (or other oxidizing gas) can be predetermined, and the number of cycles of the evacuation/aeration process discussed above can be adjusted accordingly to achieve the desired final concentration. For example, in one embodiment, the method of controlling oxygen content in the headspace of the sealed drug product container includes: (a) determining a desired final oxygen content in the headspace of the sealed container; (b) calculating an end % oxygen content following a first cycle of oxygen reduction via equation (I):

$$\% {O}_{2 , end} = ( \frac{{P}_{vacuum}}{{P}_{aeration}} ) * \% {O}_{2 , start} \qquad ( I )$$

wherein % O_{2, start} is the % oxygen content at the start of the first cycle, P_{vacuum} is an evacuation pressure applied in the first cycle of oxygen reduction, Pₐₑᵣₐₜᵢₒₙ is a pressure higher than P_{vacuum} but less than 1 bar, and % O_{2. end} is the % oxygen content at the end of the first cycle; (c) optionally applying equation (I) to additional cycles, wherein % O_{2, start} is the % oxygen content at the end of the preceding cycle, until the desired final oxygen content is reached; and (d) preparing a drug product in the sealed container by: (i) performing one or more cycles of oxygen reduction by evacuating an unsealed container in a vacuum chamber at P_{vacuum}, wherein P_{vacuum} is a pressure of from 0.05 bar to 0.15 bar, and aerating the unsealed container in the vacuum chamber with a non-oxidizing gas at an aeration pressure of from 800 mbar to 1000 mbar; and (ii) sealing the container.

In various embodiments, the pressure is maintained above the vapor pressure of water to avoid evaporation of the liquid formulation containing the recombinant proteins or antigen-binding proteins. In various embodiments, evacuation and/or aeration of the vacuum chamber occurs at a pressure of from about 0.02 bar to about 0.2 bar. In one embodiment, evacuation occurs at a pressure of about 0.1 bar, and aeration occurs at a pressure of from about 800 mbar to about 1000 mbar. The non-oxidizing gas used in the aeration of the vacuum chamber can be selected from, e.g., nitrogen, argon, helium, xenon, neon, krypton and radon. In one embodiment, the non-oxidizing gas is nitrogen. In another embodiment, the non-oxidizing gas is argon. In various embodiments, the methods are performed at a temperature in a range of from about 5-45° C. or in a range of from about 10-37° C. In various embodiments, the methods are performed at a temperature in a range of from about 15-25° C. In some cases, the temperature is about 15° C., about 16° C., about 17° C., about 18° C., about 19° C., about 20° C., about 21° C., about 22° C., about 23° C., about 24° C., or about 25° C. In one embodiment, the temperature of all cycles is maintained at about 19° C.

The recombinant proteins or antigen-binding protein compositions sealed within the drug product containers in accordance with the methods of the present invention can be any of the various compositions discussed above or herein. For example, the liquid compositions of the antigen-binding proteins (e.g., antibodies) can be formulated with various excipients, including buffers, tonicity modifiers, stabilizers, surfactants and the like, and the proteins can be present at concentrations ranging from about 0.1 mg/ml to about 200 mg/ml. In some embodiments, the concentration of the antibody or other antigen-binding protein is from about 1 mg/ml to about 25 mg/ml, from 1 mg/ml to about 15 mg/ml, or from about 1 mg/ml to about 10 mg/ml. In some cases, the concentration is less than 10 mg/ml, less than 5 mg/ml, less than 2 mg/ml or less than 1 mg/ml.

As discussed above, after reducing the oxidizing gas content of the headspace gas within the container(s), the container(s) are fully closed/stoppered and finally sealed. The stopper can be made of a variety of materials (e.g. polymer, rubber) and exhibit elastomeric properties (e.g. sufficient rigidity, malleability) as desired for engagement with the container. In some embodiments the stopper is formed of synthetic rubber In some embodiments the stopper is formed of butyl rubber. The stopper may comprise one or more vents, or vent legs. The stopper can be adapted to form and retain an elastomeric seal and can, in some embodiments, be a stopper adapted to conventional lyophilization procedures. As such, a stopper for use in a vial in a lyophilization chamber may be partially stoppered with the vent(s) open to the outside airspace during the gas overlay/oxygen reduction process, and then fully stoppered and sealed in connection with the container following the one or more oxygen reduction cycles. Examples of lyophilization systems, closure cap and stopper configurations are provided in FDA Guide “Lyophilization of Parenteral (7/93)’ and Bhambhani and Medi, “Selection of Containers/Closures for Use in Lyophilization Applications: Possibilities and Limitations,” American Pharmaceutical Review, May 1, 2010, the contents of which are hereby incorporated by reference in their entirety.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Reduction of Oxygen in Drug Product Headspace

In this example, a standard GMP lyophilization chamber, fitted with a Pirani vacuum gauge to measure, and a needle valve to control the pressure, was used as the vacuum chamber. A bispecific antibody at a concentration of 2 mg/ml in a liquid formulation was packaged in a vial fitted with a vent-leg rubber stopper with nitrogen overlay in a two-cycle process to reduce the presence of headspace oxygen from ˜21% to about 0.25%.

TABLE 1
Non-Oxidizing Gas Overlay Process

| | | SHELF | | | |
|---|---|---|---|---|---|
| | | TEMP | SHELF | | |
| | | HOLD/ | TEMP | | STEP |
| STEP | | RAMP | RAMP | CHAMBER | TIME |
| No. | PHASE | (° C.) | RATE | PRESSURE | (HH:mm) |
| 1 | Start | N/A | N/A | N/A | N/A |
| 2 | Door Lock/ | N/A | N/A | N/A | N/A |
| | Unlock | | | | |
| 3 | Aeration | N/A | N/A | to +0.02 | N/A |
| 4 | Loading | +19 | N/A | Atmospheric | 00:01 |
| 5 | Stabilization | +19 | N/A | Atmospheric | 00:05 |
| 6 | Vacuum | +19 | N/A | 100, mbar (Pirani, | 00:01 |
| | Initiation | | | Vacuum Valves | |
| | | | | control) | |
| 7 | Aeration | +19 | N/A | 912 mbar | N/A |
| | (N2) | | | | |
| 8 | End | N/A | N/A | N/A | N/A |

Once the vials were placed in the chamber, vacuum was pulled (step 6) to remove the gas (which to start out is air containing ˜21% oxygen) from the chamber. The pressure (100,000 μbar) is above the vapor pressure of water to avoid evaporation, foam formation and potential splash, and is measured with the Pirani gauge. Under ordinary lyophilization conditions, a vacuum to much lower pressures (on the order of 150 μbar) is present, so pressure is controlled using a capacitance manometer. However, the capacitance manometer is only accurate at pressures below about 2000 μbar, and cannot be used to control the pressure at 100,000 μbar.

After reaching the 100,000 μbar pressure, nitrogen was filled into the chamber replacing the evacuated air.

The process was repeated a second time to further reduce oxygen levels (2^{nd} Cycle Steps 3-4 below). Once the desired oxygen level was reached, the vials were stoppered (2^{nd} Cycle Step 5 below).

TABLE 2
Non-Oxidizing Gas Overlay Process, Continued

| | | SHELF | | | |
|---|---|---|---|---|---|
| | | TEMP | SHELF | | |
| | | HOLD/ | TEMP | | STEP |
| STEP | | RAMP | RAMP | CHAMBER | TIME |
| No. | PHASE | (° C.) | RATE | PRESSURE | (HH:mm) |
| 1 | Start | N/A | N/A | N/A | N/A |
| 2 | Stabilization | +19 | N/A | Atmospheric | 00:05 |
| 3 | Stabilization | +19 | N/A | 100 mbar (Pirani, | 00:01 |
| | | | | Vacuum Valves | |
| | | | | control) | |
| 4 | Pre-aeration | +19 | N/A | 912 mbar | 00:01 |
| | (N₂) | | | | |
| 5 | Stoppering | +19 | N/A | 912 mbar | (130 bar |
| | | | | | for 30 sec) |
| 6 | Unloading | +19 | N/A | Atmospheric | N/A |
| 7 | N/A | N/A | N/A | N/A | N/A |

Oxygen Content Equation:

$$\% {O}_{2 , end} = ( \frac{{P}_{vacuum}}{{P}_{aeration}} ) * \% {O}_{2 , start}$$

$$P = Pressure$$

Example calculation, from the process discussed above in Example 1:

$$Cycle 1$$

$${P}_{vacuum} = 100 mbar$$

$${P}_{aeration} = 912 mbar$$

$$\% {O}_{2 , start} = 21 \% ( Starting with air in the chamber )$$

$$\% {O}_{2 , end} = ( \frac{100 mbar}{912 mbar} ) * 21 \%$$

$$\% {O}_{2 , end} = 2.3 \%$$

$$Cycle 2$$

$${P}_{vacuum} = 100 mbar$$

$${P}_{aeration} = 900 mbar$$

$$\% {O}_{2 , start} = 2 \%$$

$$\% {O}_{2 , end} = ( \frac{100 bar}{912 mbar} ) * 2.3 \%$$

$$\% {O}_{2 , end} = 0.25 \%$$

### Example 2: Stability Testing of Drug Products

Stability analyses were performed on various drug products prepared using the process discussed in Example 1, with varying concentrations of headspace oxygen, and compared against controls in which headspace oxygen was at or near atmospheric levels (˜21%). In some cases (noted herein) nitrogen was replaced with argon in the aeration portions of the process. High molecular weight (HMW) species were detected using size exclusion ultra performance liquid chromatography (SE-UPLC), and charge variant species were detected using cation exchange ultra performance liquid chromatography (CEX-UPLC).

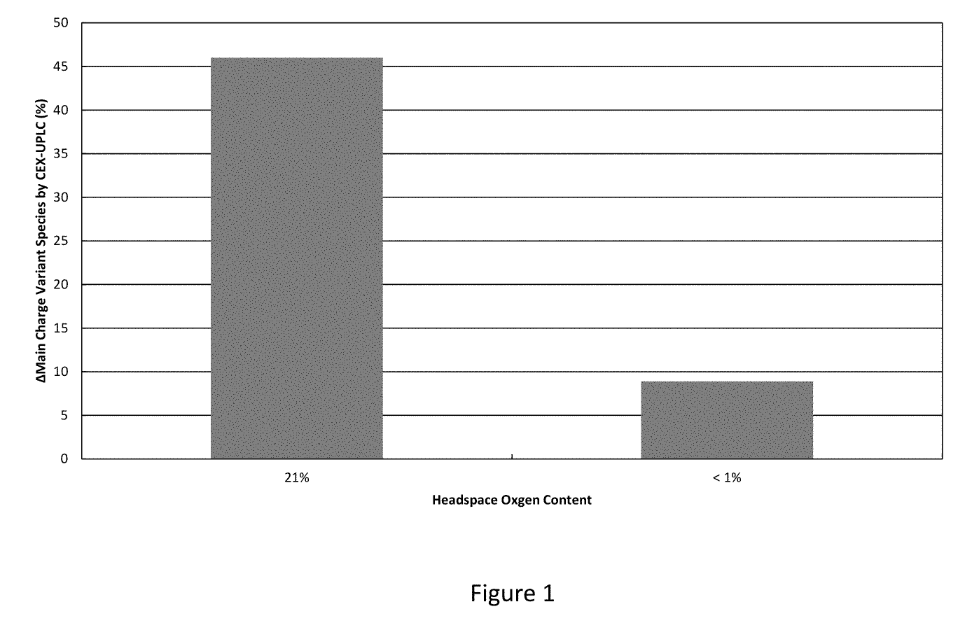

As illustrated in FIG. 1, reducing the headspace oxygen content, via a nitrogen overlay, from 21% to less than 1% reduced degradation of the bispecific antibody observed by CEX-UPLC following 31 months of storage at 5° C. At 21% oxygen, the percentage change in main charge variant species was about 46.25%, whereas at <1% oxygen, the percentage change was reduced to about 8.75% over the storage period.

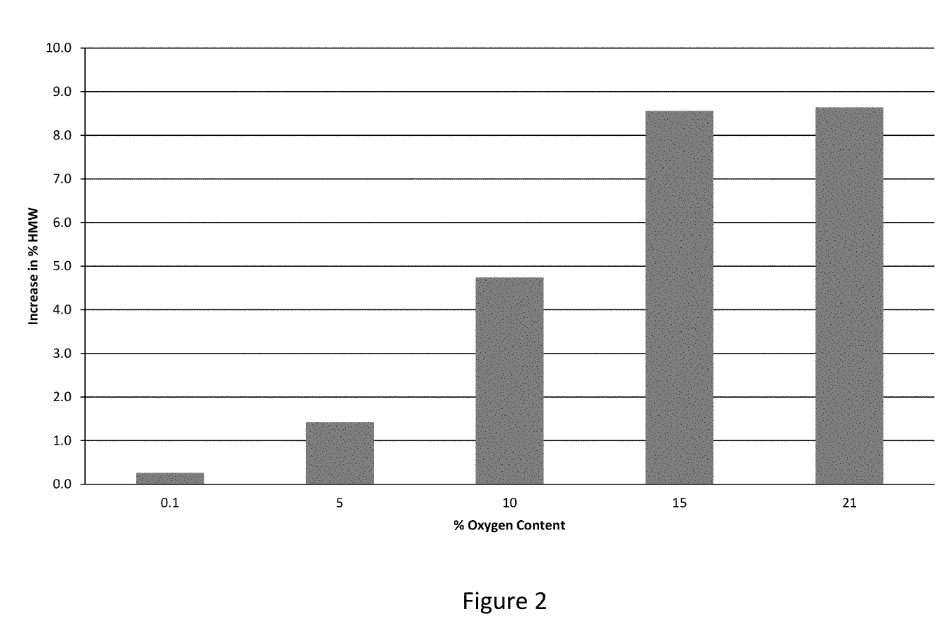

As illustrated in FIG. 2, reducing the headspace oxygen content, via a nitrogen overlay, from 21% to 0.1% increased the stability of a second bispecific antibody, as shown by the reduction in percentage increase in the presence of HMW species, following 28 days of storage at 45° C. At 21% oxygen, there was an increase in the percentage of HMW species of about 8.62% over the storage period. At 15% oxygen, there was an increase in the percentage of HMW species of about 8.53% over the storage period. At 10% oxygen, there was an increase in the percentage of HMW species of about 4.74% over the storage period. At 5% oxygen, there was an increase in the percentage of HMW species of about 1.42% over the storage period, and at 0.1% oxygen, there was an increase in the percentage of HMW species of about 0.24% over the storage period.

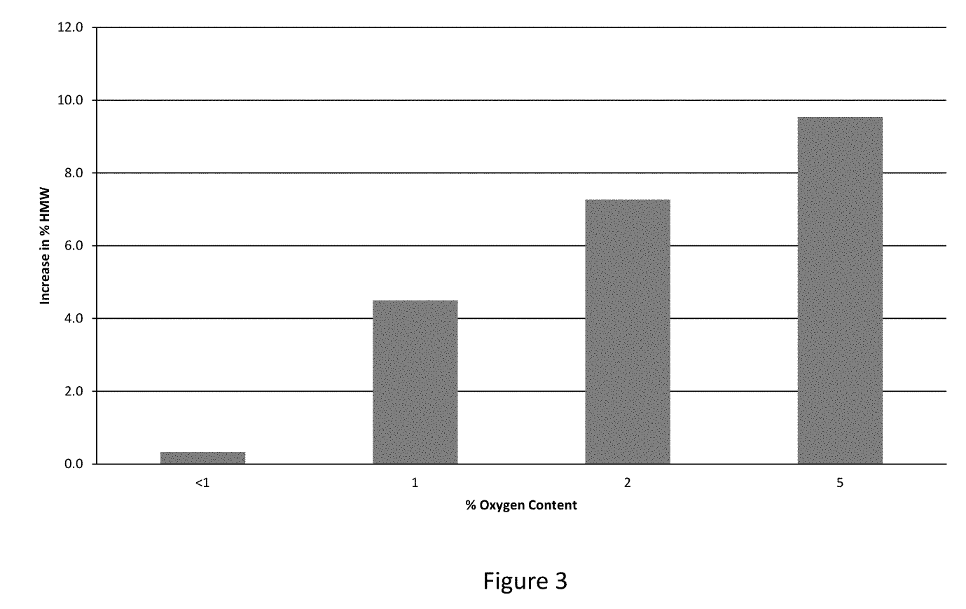

FIG. 3 illustrates the reduction in percentage increase in the presence of HMW species for the second bispecific antibody observed after three months of storage at 45° C., following the nitrogen overlay process to reduce the headspace oxygen content. At 5% oxygen, there was an increase in the percentage of HMW species of about 9.66% over the storage period. At 2% oxygen, there was an increase in the percentage of HMW species of about 7.27% over the storage period. At 1% oxygen, there was an increase in the percentage of HMW species of about 4.54% over the storage period, and at less than 1% oxygen, there was an increase in the percentage of HMW species of about 0.34% oxygen over the storage period.

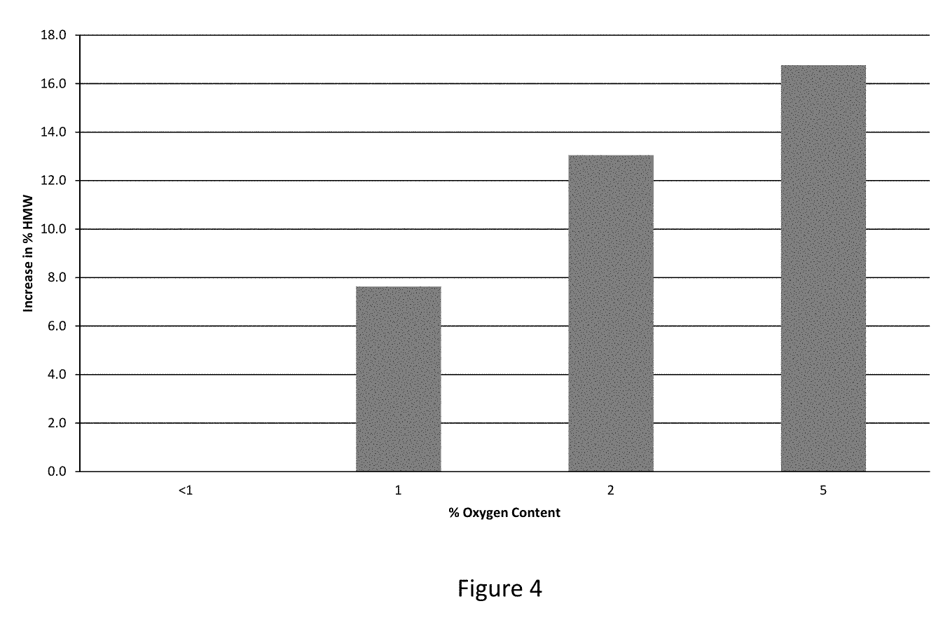

FIG. 4 illustrates the same headspace oxygen content for the second bispecific antibody stored at 45° C. for three months as in FIG. 3, except the nitrogen in the overlay process was replaced with argon. At 5% oxygen, there was an increase in the percentage of HMW species of about 16.72% over the storage period. At 2% oxygen, there was an increase in the percentage of HMW species of about 13.05% over the storage period. At 1% oxygen, there was an increase in the percentage of HMW species of about 7.68% over the storage period, but at less than 1% oxygen, there was no detectable increase in HMW species over the storage period.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

1. A stable liquid drug product comprising a sealed container containing a recombinant protein in a liquid formulation and a headspace comprising a gas, wherein the gas comprises less than 5% oxygen by volume and the recombinant protein is stable for a period of at least 28 days when stored at 45° C., wherein stable for at least 28 days refers to (i) an increase in percentage of high molecular weight species of no more than 2% over the period, or (ii) a change in at least one product CQA above or below a predefined threshold.

2. The drug product of claim 1, wherein the gas comprises no more than 2% oxygen by volume, no more than 1% oxygen by volume, less than 1% oxygen by volume, or no more than 0.1% oxygen by volume.

3-5. (canceled)

6. The drug product of claim 1, wherein the liquid formulation contains the recombinant protein at a concentration of less than 10 mg/ml, less than 5 mg/ml, or less than 2 mg/ml.

7-8. (canceled)

9. The drug product of claim 1, wherein the liquid formulation contains the recombinant protein at a concentration of from 1 mg/ml to 200 mg/ml.

10. The drug product of claim 1, wherein the recombinant protein is stable for a period of at least three months when stored at 45° C., wherein stable for at least three months refers to an increase in percentage of high molecular weight species of no more than 10%, no more than 5%, or no more than 1%, over the period.

11-13. (canceled)

14. The drug product of claim 15, wherein the recombinant protein is stable for a period of at least 31 months when stored at 5° C., wherein stable for at least 31 months refers to a change in percentage of main charge variant species of no more than 10% over the period.

15. A drug product comprising a sealed container containing a recombinant protein in a liquid formulation and a headspace comprising a gas, wherein the gas comprises less than 1% oxygen by volume.

16. The drug product of claim 15, wherein the gas comprises no more than 0.1% oxygen by volume.

17. The drug product of claim 1, wherein the recombinant protein is an antigen-binding protein.

18. The drug product of claim 17, wherein the antigen-binding protein is a monospecific antibody or a bispecific antibody.

19-20. (canceled)

21. The drug product of claim 1, wherein the sealed container is a vial comprising a stopper having one or more vent legs.

22. A method of preparing a drug product in a sealed container containing a recombinant protein in a liquid formulation and a headspace comprising a gas with reduced oxygen content, the method comprising:
(a) loading one or more containers containing the liquid formulation of the recombinant protein into a vacuum chamber under atmospheric pressure;
(b) evacuating the chamber at a first pressure of from 0.05 bar to 0.15 bar;
(c) aerating the chamber with a non-oxidizing gas at a second pressure greater than the first pressure but less than 1 bar; and
(d) sealing the one or more containers,
wherein the method is performed at a temperature in a range of from 5-45° C., and the sealed container comprises a headspace gas with less than 5% oxygen by volume.

23. The method of claim 22, further comprising repeating steps (b) and (c) one or more additional times prior to sealing the one or more containers.

24. The method of claim 22, wherein the first pressure is about 0.1 bar, and the second pressure is from about 800 mbar to about 1000 mbar.

25. (canceled)

26. The method of claim 22, wherein the temperature is in a range of from about 15-25° C., optionally wherein the temperature is about 19° C.

27. (canceled)

28. The method of claim 22, wherein the sealed container comprises a headspace gas with less than 2% oxygen by volume, less than 1% oxygen by volume, or less than 0.1% oxygen by volume.

29-30. (canceled)

31. The method of claim 22, wherein the liquid formulation contains the recombinant protein at a concentration of from 1 mg/ml to 200 mg/ml.

32. The method of claim 22, wherein the liquid formulation contains the recombinant protein at a concentration of less than 10 mg/ml, less than 5 mg/ml, or less than 2 mg/ml.

33-34. (canceled)

35. The method of claim 22, wherein the recombinant protein is an antigen-binding protein.

36. The method of claim 35, wherein the antigen-binding protein is a monospecific antibody, or a bispecific antibody.

37-38. (canceled)

39. The method of claim 22, wherein the sealed container is a vial comprising a stopper having one or more vent legs.

40. The method of claim 39, wherein the one or more vent legs is partially closed during step (b) and/or step (c), or the one or more vent legs is fully closed during step (d).

41. (canceled)

42. The method of claim 22, wherein the pressure in the chamber is measured via a Pirani vacuum gauge.

43. The method of claim 22, wherein the non-oxidizing gas is nitrogen or argon, or is selected from the group consisting of helium, xenon, neon, krypton and radon.

44-45. (canceled)

46. A method of controlling oxygen content in a headspace of a sealed container containing a liquid pharmaceutical formulation, comprising:
(a) determining a desired final oxygen content in the headspace of the sealed container;
(b) calculating an end % oxygen content following a first cycle of oxygen reduction via equation (I):
$$\% {O}_{2 , end} = ( \frac{{P}_{vacuum}}{{P}_{aeration}} ) * \% {O}_{2 , start} \qquad ( I )$$
wherein % O_{2, start} is the % oxygen content at the start of the first cycle, P_{vacuum} is an evacuation pressure applied in the first cycle of oxygen reduction, Pₐₑᵣₐₜᵢₒₙ is a pressure higher than P_{vacuum} but less than 1 bar, and % O_{2. end} is the % oxygen content at the end of the first cycle;
(c) optionally applying equation (I) to additional cycles, wherein % O_{2, start} is the % oxygen content at the end of the preceding cycle, until the desired final oxygen content is reached; and
(d) preparing a drug product in the sealed container by: (i) performing one or more cycles of oxygen reduction by evacuating an unsealed container in a vacuum chamber at P_{vacuum}, wherein P_{vacuum} is a pressure of from 0.05 bar to 0.15 bar, and aerating the unsealed container in the vacuum chamber with a non-oxidizing gas at an aeration pressure of from 800 mbar to 1000 mbar; and (ii) sealing the container.

47. A drug product comprising a sealed vial containing a recombinant protein in a liquid formulation and a headspace comprising a gas, wherein the drug product is prepared by the method of claim 39.
